(19) United States
(12) Patent Application Publication
(10) Pub. No.: US 2026/0165918 A1
(43) Pub. Date: Jun. 18, 2026

(54) HAIR DYE COMPOSITION HAVING IMPROVED VISCOSITY

(71) Applicant: JMM LIMITED, Hong Kong (CN)

(72) Inventors: Tsz Siu CHOW, Hong Kong (CN); Ying Lok MAN, Hong Kong (CN); Man Ho MOK, Hong Kong (CN); Ching Man CHAN, Hong Kong (CN)

(21) Appl. No.: 19/126,616

(22) PCT Filed: Sep. 19, 2022
(86) PCT No.: PCT/CN2022/119652

§ 371 Date: May 1, 2025

(51) Int. Cl. A61K 8/19; A61K 8/11; A61K 8/41; A61Q 5/06; A61Q 5/10; C01B 32/23

(52) U.S. Cl. CPC A61K 8/19; A61K 8/11; A61K 8/41; A61K 8/416; A61Q 5/065; A61Q 5/10; C01B 32/23

(57) ABSTRACT

The present disclosure lies in the field of cosmetics. In particular, the present disclosures relate to hair dye compositions (waterborne hair coating solution) having improved viscosity. A feature here of these compositions is that such compositions comprise complex selected from iron-tannic acid complex, iron-gallic acid complex or mixture thereof.

### TECHNICAL FIELD

The present disclosure lies in the field of cosmetics. In particular, the present disclosures relate to hair dye compositions (waterborne hair coating solution) having improved viscosity.

### BACKGROUND ART

Traditional permanent hair dyes have several obvious disadvantages, including:
(1) They destroy hair textures;
(2) They cause potential risks to human health;
(3) They bring possible environmental problems in disposal;
(4) Both customers and stylists suffer from long treatment time.

CN106477559A relates to modified graphene hair dye, including coloring agent and color developing agent. The coloring agent includes modified graphene, gallic acid, auxiliary agent, cosolvent, distilled water. Said graphene plays a role of far-infrared physiotherapy hair care after hair dyeing. However, said prior art is silent to the use of any iron-tannic acid complex or iron-gallic acid complex and such prior art is silent to the problem of viscosity of the graphene hair dye as well as the technical solution to solve such problem.

CN106477559A relates to graphene and preparation method thereof. In the method of this prior art, gallic acid and tannic acid are added in the modified graphene oxide dispersion liquid to obtain final product graphene. However, said prior art is silent to the use of any iron-tannic acid complex or iron-gallic acid complex and said prior art is silent to the problem of viscosity of the graphene hair dye as well as the technical solution to solve such problem.

Although there are alternatives to traditional permanent hair dyes in the market, these alternatives suffer from several drawbacks during application. Some semi-permanent hair dyes do not contain irritants like ammonia and hydrogen peroxide, but they still contain carcinogen p-phenylenediamine (PPD). Secondly, there are hair dyes in the market that claim to be natural-based and hence less harmful, but it was reported that these natural-based hair dyes actually contain toxic heavy metals. For temporary hair color spray products which allowed a fast treatment time, its application is through spraying of a color solution with low viscosity and the coloring effect is not long lasting.

As a result, there is a great industry demand for an easily applied, safe, environmentally friendly, and long lasting hair dye product.

### CONTENTS OF INVENTION

One of the subjects of the present disclosure is hair dye compositions. A feature here of these compositions is that such compositions comprise complex selected from iron-tannic acid complex, iron-gallic acid complex or mixture thereof.

A further subject of the present disclosure is the use of complex selected from iron-tannic acid complex, iron-gallic acid complex or mixture thereof defined in the composition according to the preceding claims for increasing the viscosity of the composition.

A further subject is also a method for dying hair in which the above-described composition is used.

Other features, elements, steps, characteristics and advantages of the present invention will become more apparent from the following detailed description of preferred embodiments of the present invention.

### DESCRIPTION OF DRAWING

FIG. 1 shows the structure of (a) graphene, (b) graphene oxide and (c) reduced graphene oxide.

FIG. 2 shows the difference between functionalized graphene and other pigments and dyes on hair.

FIG. 3 shows the proposed x-x interaction between functionalized graphene and tannic acid ligands.

FIG. 4 shows the operation process of waterborne hair coating solution.

FIG. 5 shows the comparison of the viscosity between hair coating solution with/without iron(III) ion.

### SPECIFIC MODE FOR CARRYING OUT THE INVENTION

The present invention provides a safe and eco-friendly waterborne coating for coloring hair comprising functionalized graphene as the coloring component. Such non-invasive coating solution for dyeing hair will not cause damage to the hair structure. Also, it is formulated with non-harmful components into water-based (waterborne) solution for a safe and environmentally friendly product. The hair dye process can be as fast as 10 minutes, a drastic time saving from the long treatment time (1 hour) in traditional hair coloring treatment.

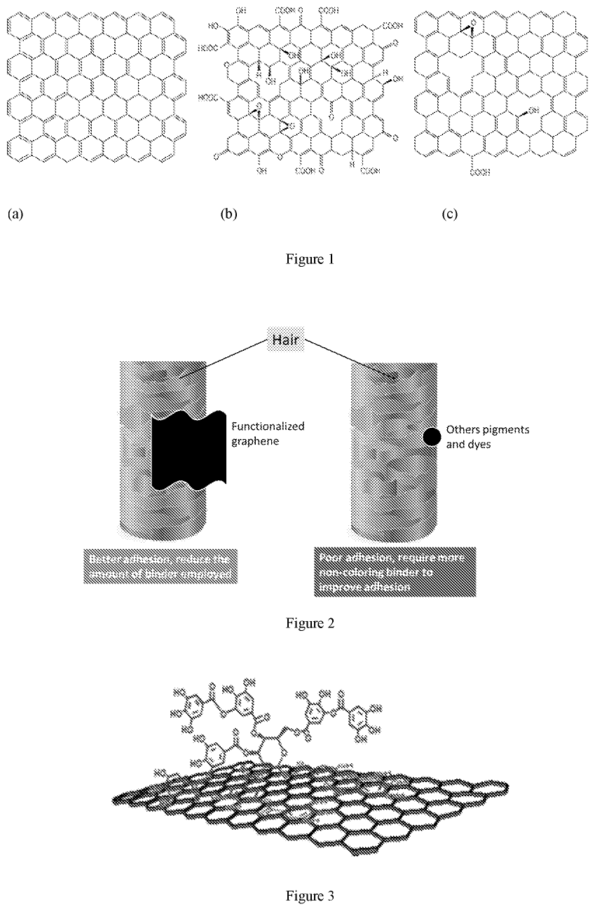

The waterborne hair coating employs functionalized graphene as the coloring component. Graphene is composed of carbon atoms, arranged in a hexagonal lattice to form single-atom-thick, two-dimensional sheets. The structures of graphene and some functionalized graphene are shown in FIG. 1. Graphene is exclusive composed of carbon atom element in the whole structure. However, upon different preparation methods of graphene from different precursors, the resultant prepared graphene will be consisted of different additional functional groups (which do not necessary composed of carbon atom element) as well. Some examples of the mentioned functional groups included hydroxyl group and carboxylic acid group. With different functional groups present in the graphene structure, different properties such as colours and interactions between the functional graphene-polymer coating solutions will be resulted. For example, the reduced graphene oxide (rGO) mentioned in the procedure example consist of hydroxyl group (—OH) in the graphene structure, is prepared by reducing graphene oxide precursor by ascorbic acid.

The extended two-dimensional sheets structure of functionalized graphene provided a large surface area to attach the hair surface well hence providing better adhesion without using a lot of adhesion promoting binder additives. On the other hand, most of the pigments and dyes do not have such 2D sheet structure and cannot deliver such adhesion as functionalized graphene, c.f., FIG. 2.

For ease of application and long lasting effect, the viscosity of waterborne hair coating solution is increased with the addition of iron-tannic acid and iron-gallic acid complexes. These complexes also promote the dispersion of functionalized graphene inside waterborne solution. Such effect can be rationalized by the T-interaction between functionalized graphene and tannic acid or gallic acid ligands (FIG. 3). It is theoretically expected that said x-x interaction is between the T-electron clouds of graphene structure and the x-electron clouds of the hydroxylated phenyl group in both tannic acid and gallic acid. For the interaction between iron-gallic acid, the iron(III) ion is expected to be interacted with the hydroxyl group of the gallic acid in the hair coating solution.

Unless otherwise depicted, the viscosity of samples including the prepared hair coating solution according to the present invention as well as other comparative samples in the art are tested by viscometer. Ametek Brookfield DVIM viscometer is employed for measurement. It can measure solution in viscosity range between 15-2000000 cP. During measurement of the viscosity, the selected spindle was put into a beaker of samples to be tested and the viscosity of the samples will be measured under different spin rate. Viscosity of the samples under the same spin rate is thus compared.

One of the differences between the graphene hair dye of the prior art and graphene hair coating solution according to the present invention is the viscosity of the solution formed. The presence of iron-tannic acid and/or iron-gallic acid complexes additives in the formulation creates a solution texture similar to traditional hair dye solutions, providing familiarity to the hair stylists during treatment. The role of iron (in the form of iron(III)) in the solution is to interacting with tannic acid/gallic acid to furnish a solution with higher viscosity.

With such improved adhesion and viscosity by the effects of both functionalized graphene and iron-tannic acid/gallic acid complexes, the waterborne hair coating solution according to the present invention can be applied on the hair in 10 minutes treatment time, which is much faster than the nearly 1 hour treatment for conventional hair coloring treatment. As a result, this waterborne hair coating is expected to bring a revolution to the hair coloring market.

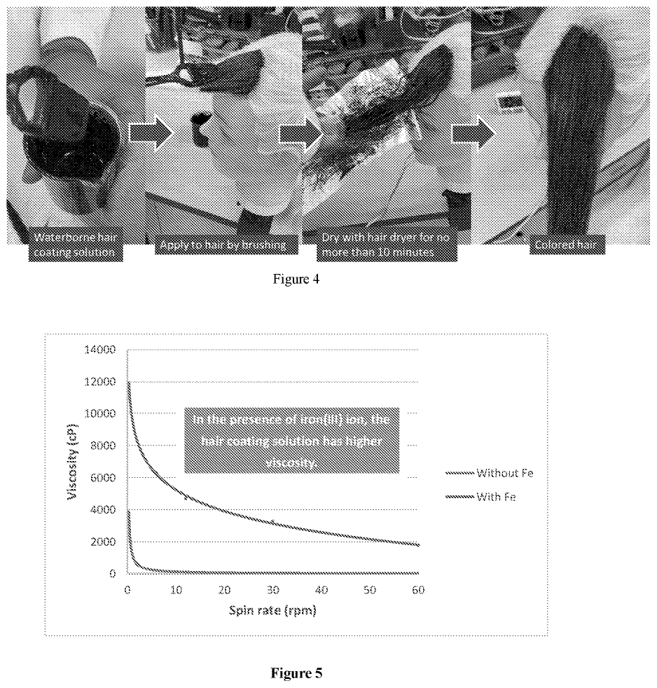

With the waterborne hair coating solution according to the present invention on hand, it can be applied onto human hair using the same brushing method of the traditional hair coloring dyes do (FIG. 4).

The waterborne hair coating solution according to the present invention can be prepared by mixing graphene oxide solution, L-ascorbic acid, tannic acid, gallic acid, iron(III) nitrate nonahydrate, deionised water/distilled water.

Non-limiting Example of the compositions invention are as follows:

### Example 1

### Preparation and Application of Waterborne Hair Coating Solution (Black)

### 1. Materials

2.5 wt % Graphene oxide solution, Brand: Graphenea
L-Ascorbic acid, CAS no. 50-81-7, Brand: TCI
Tannic acid, CAS no. 1401-55-4, Brand: Sigma Aldrich
Gallic acid, CAS no. 5995-86-8, Brand: TCI
Iron(III) nitrate nonahydrate, CAS no. 7782-61-8, Brand: Sigma Aldrich
Deionised water/distilled water

### 2. Personal Protective Equipment

Gloves
Safety goggles
Mask
Laboratory coating

### 3. Equipment and Apparatus

Weighing scale, Shimadzu
Hotplate stirrer, IKA
Sonicator with probe, Qsonica
Air condenser, Chemglass Life Sciences
Magnetic stirring bar
Round bottom flask
Büchner funnel with conical flask
Beaker
Automatic pipette

### 4. Procedure

4.1 Synthesis of Reduced Graphene Oxide (rGO) Paste:

4.1.1 40 g of 2.5 wt % graphene oxide solution, 1 g of tannic acid, 10 g of ascorbic acid and 100 g of deionised water/distilled water are added into 500 mL round bottom flask with a magnetic stirring bar.

4.1.2 The flask is connected with an air condenser and heated under 120° C. for 1 hour.

4.1.3 The reaction mixture is filtered by suction filtration and rGO paste is obtained as residue.

4.1.4 The rGO paste is washed with DI water for several times until the filtrate has become clear and its pH has reached to 7.

### 4.2 Synthesis of Black Colour Waterborne Hair Coating Solution:

4.2.1 Top up the as-prepared rGO paste (obtained from procedure 4.1.4) with deionised water/distilled water to 54 g followed by the addition of 0.5 g of gallic acid. The whole solution is mixed homogeneously under ultra-sonication for 1 min (Process time: 30 s pulse on and 30 s pulse off)

4.2.2 5.94 mL of 0.4 g/mL iron(III) nitrate nonahydrate aqueous solution (prepared by mixing 4 g iron(III) nitrate nonahydrate with deionised water/distilled water until a 10 mL solution is formed) is added into the mixture. The whole solution is further mixed homogeneously under ultra-sonication for 1 min (Process time: 30 s pulse on and 30 s pulse off)

### 4.3 Dye Application Process

4.3.1 The black colour waterborne hair coating solution (obtained from procedure 4.2.2) is applied on the hair, then dried using hot air setting with a hair dryer for 4 min

4.3.2 The black colour waterborne hair coating solution is applied on the hair again, then dried using hot air setting with a hair dryer for 8 min

4.3.3 The coated hair is allowed to set in air for 30 min

4.3.4 The coated hair is shampooed, washed thoroughly with tap water and dried using a hair dryer

### Example 2

The graphene hair dyeing solution according to the present invention as prepared above is compared with four prior art hair dyes:

### Traditional Permanent Hair Dye;

Example: L'Oreal Paris Excellence Creme Permanent Hair Color; ingredients and detailed information: please refer to https://www.lorealparisusa.com/products/hair-color/products/permanent/excellence-cr eme-permanent-triple-protection-hair-color.aspx?shade=4-dark-brown

### Semi-Permanent Hair Dye

Example: L'Oreal Paris Casting Crème Gloss Semi-Permanent Hair Dye, Ammonia-Free Formula & Honey-Infused Conditioner, Glossy Finish; ingredients and detailed information: please refer to https://www.amazon.co.uk/LOreal-Casting-Semi-Permanent-leaving-radiant/dp/B087 XLP661

### Nature-Based Hair Dye

Example: Vatika Henna Hair Colour Natural-Black; ingredients and detailed information: please refer to https://www.tesco.com/groceries/en-GB/products/294497170

### Temporary Hair Coloring Sprays

Example: B-Wild Temporary Hair Color Spray; ingredients and detailed information: please refer to https://www.walgreens.com/store/c/b-wild-temporary-hair-color-spray/ID=prod9333-product

The test results are shown in the following Table.

TABLE

| | | | | Temporary | NAMI-JMM |
|---|---|---|---|---|---|
| Hair | Traditional | | Natural | Hair | Waterborne |
| Coloring | Permanent | Semi-permanent | Material-based | Coloring | Hair |
| Methods | Hair Dye | Hair Dye | Hair Dye | Sprays | Coating |
| Destroying | Yes, by | Yes, but less | Yes, by hair | No | No |
| hair | opening | than permanent | damaging | | |
| structure | the cuticle | hair dye | additive(s) | | |
| | layer and | | | | |
| | enter into | | | | |
| | the cortex | | | | |
| Risk to | Yes | Yes | Yes | No | No |
| human | | | | | |
| health | | | | | |
| Durability | >50 | 4-8 | 4-6 | 1-4 | 20-50 |
| (hair wash | | | | | |
| cycles) | | | | | |

A fast, safe, and eco-friendly waterborne hair coating solution has been prepared by using functionalized graphene as the coloring component, with short treatment time and long lasting color retention without damaging the hair texture.

The Inventor has found a non-invasive coating formulation for hair dyeing without employing harmful chemicals such as ammonia, hydrogen peroxide, and carcinogenic p-phenylenediamine (PPD) additives, which are commonly used in traditional hair dye formulation. Functionalized graphene, consisting of extended two-dimensional sheets structure, acts as the coloring agent of this waterborne hair coating. Such sheet structure provides a large surface area to cover the entire hair surface, providing strong adhesion and durability. The presence of iron-tannic acid and iron-gallic acid complexes additives in the formulation creates a solution texture similar to traditional hair dye solutions, providing familiarity to the hair stylists during treatment. Most importantly, the developed waterborne hair coating solution can cut the treatment time from the traditional 1 hour to only 10 minutes, a huge impact to the hair coloring industry.

### Example 3

The graphene hair dyeing solution according to the present invention as prepared above as well as graphene hair dye of the prior art are tested for their viscosity with the above mentioned viscometer.

FIG. 5 shows the test results available for viscosity between hair coating solution with/without iron(III) ion was compared.

It is found that in the presence of iron(III) ion of the iron-tannic acid/iron-gallic acid complex according to the present invention, the hair coating solution has significantly higher viscosity.

Also, the viscosity of the graphene hair dye of the prior art has been measured, and it is found that the viscosity is below 15 cP when the spin rate is higher than 10 rpm. The extremely low viscosity of the prior art formulation will retard its application under real situation.

The iron-tannic acid/iron-gallic acid complex in the hair coating solution is found to be most durable and its viscosity is most suitable for hair coating application.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the protection scope of the various embodiments.

1. Hair dye composition comprising graphene and complex selected from iron-tannic acid complex, iron-gallic acid complex or mixture thereof.

2. The composition according to claim 1, wherein the graphene is reduced graphene oxide.

3. The composition according to claim 1 or 2, wherein the reduced graphene oxide comprises hydroxyl group in the graphene structure.

4. The composition according to claim 1 or 2, wherein the complex is a mixture of iron-tannic acid complex, iron-gallic acid complex.

5. The composition according to claim 1 or 2, wherein the iron in the complex is in the form of iron(III).

6. The use of complex selected from iron-tannic acid complex, iron-gallic acid complex or mixture thereof defined in the composition according to the preceding claims for increasing the viscosity of the composition.

7. A method for dying hair, wherein the composition according to the preceding claims is used.
